(19) United States
(12) Plant Patent Application Publication
(10) Pub. No.: US 2015/0020248 P1
(43) Pub. Date: Jan. 15, 2015

(54) Strawberry plant named "Sahara"

(50) ;

(71) Applicant: Alexandre Pierron-Darbonne, Navarra (ES)

(72) Inventor: Alexandre Pierron-Darbonne, Navarra (ES)

(21) Appl. No.: 13/987,147

(22) Filed: Jul. 3, 2013

(51) Int. Cl. A01H 5/00

(52) U.S. Cl. PLT/208

(57) ABSTRACT

A new and distinct strawberry variety, Fragaria×ananassa, cv. “Sahara” is characterized by an early time of flowering, an early time of ripening, an orange red fruit flesh color, a terminal leaflet length to width ratio that is longer than broad, and a terminal leaflet base shape that is acute.

### LATIN NAME OF THE GENUS AND SPECIES CLAIMED

Fragaria×ananassa

### VARIETY DENOMINATION

‘Sahara’

### BACKGROUND AND SUMMARY OF THE INVENTION

The present invention relates to a new and distinct strawberry variety. The varietal denomination of the new variety is “Sahara”. The new variety was designated by the breeder as “Sahara. 06.43H.79”. The new variety of strawberry was created in a breeding program by crossing two parents in 2006 in Cartaya (Huelva), Spain; in particular, by crossing as seed parent an undistributed strawberry parent designated “02-125” (unpatented) and as pollen parent an strawberry parent designated “03.98” (unpatented). Each parent is a selection from breeder's program and has not been commercialized.

The resulting seedling of the new variety was grown and asexually propagated by runners in Cartaya (Huelva), Spain, 7° W, 37° N, 45 feet elevation. Clones of the new variety were further asexually propagated and extensively tested. This propagation and testing has demonstrated that the combination of traits disclosed herein which characterize the new variety are fixed and retained true to type through successive generations of asexual reproduction.

Among the characteristics which appear to distinguish the new variety from its closest variety of which I am aware, “Sabrosa” (U.S. Plant Pat. No. 16,558) is a combination of traits which include: a terminal leaflet length to width ratio that is longer than broad for the new variety, as compared to a ratio that is as long as broad for “Sabrosa,” a terminal leaflet base shape that is acute as compared to obtuse for “Sabrosa,” a fruit flesh color that is orange red as compared to medium red for “Sabrosa,” a fruit hollow center that is weakly expressed as compared to absent or very weakly expressed for “Sabrosa,” and an early time of flowering (50% of plants at first flower) and early time of ripening (50% of plants with ripe fruit) as compared to medium times of flowering and ripening for “Sabrosa.”

The average fruit mass (g/fruit) observed for the new variety “Sahara” on March 18 was 37.9g, as compared to 39.3g for “Sabrosa” and 24.7 g for “Camarosa” (unpatented). The luminosity for the fruit of the new variety at 460nm was 18.6, as compared to 27.3 for “Sabrosa” and 31.5 for “Camarosa.”

The accompanying photographs show typical specimens of the new variety, designated “Sahara” or “06.43H.79” in the photographs.

### BRIEF DESCRIPTION OF PHOTOGRAPHS

The accompanying photographs show typical specimens of the new variety, designated “Sahara” or “06.43H.79” in the illustrations, including fruit, foliage and flower, in color as nearly true as it is reasonably possible to make in color illustrations of this character.

FIG. 1 shows several plants of the new variety “Sahara.”

FIG. 2 shows several plants of the new variety “Sahara.”

FIG. 3 shows the upper side of a complete leaf of the new variety “Sahara.” A terminal leaflet length to width ratio that is longer than broad can be seen, as can a terminal leaflet base shape that is acute. A medium green color can be seen.

FIG. 4 shows the lower side of a complete leaf of the new variety, “Sahara.” A slightly concave to flat cross-section can be seen.

FIG. 5 shows the upper side of a terminal leaflet of the new variety “Sahara.” A medium green color, and a crenate shape of incisions at the margin can be seen.

FIG. 6 shows the lower side of a terminal leaflet of the new variety “Sahara.”

FIG. 7 shows the upper side of a terminal leaflet of the new variety “Sahara” in comparison to that of “Camarosa.”

FIG. 8 shows the upper side of a terminal leaflet of the new variety “Sahara” in comparison to that of “Sabrosa.”

FIG. 9 shows the flower and calyx of the new variety, “Sahara.”

FIG. 10 shows a typical whole fruit and a sliced section of the fruit of the new variety “Sahara.” In the whole fruit, a conical shape, red color, and medium glossiness can be seen. In the sliced section, the typical flesh coloration of about orange red and a weakly expressed hollow center can be seen.

FIG. 11 shows typical whole fruit of the new variety “Sahara” in comparison to that of the variety “Camarosa.”

FIG.' 12 shows typical sliced section of the new variety “Sahara” in comparison to that of the variety “Camarosa.”

FIG. 13 shows typical whole fruit of the new variety “Sahara” in comparison to that of the variety “Sabrosa.”

FIG. 14 shows typical sliced section of the new variety “Sahara” in comparison to that of the variety “Sabrosa.”

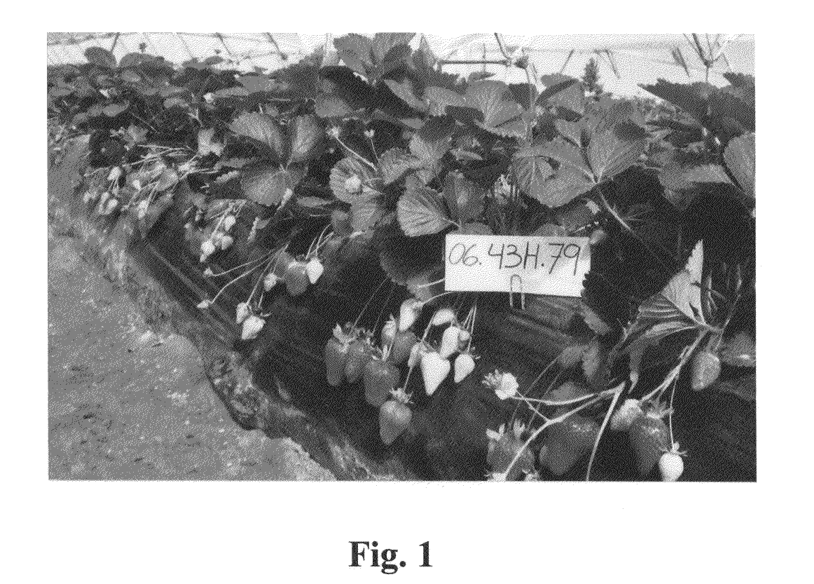

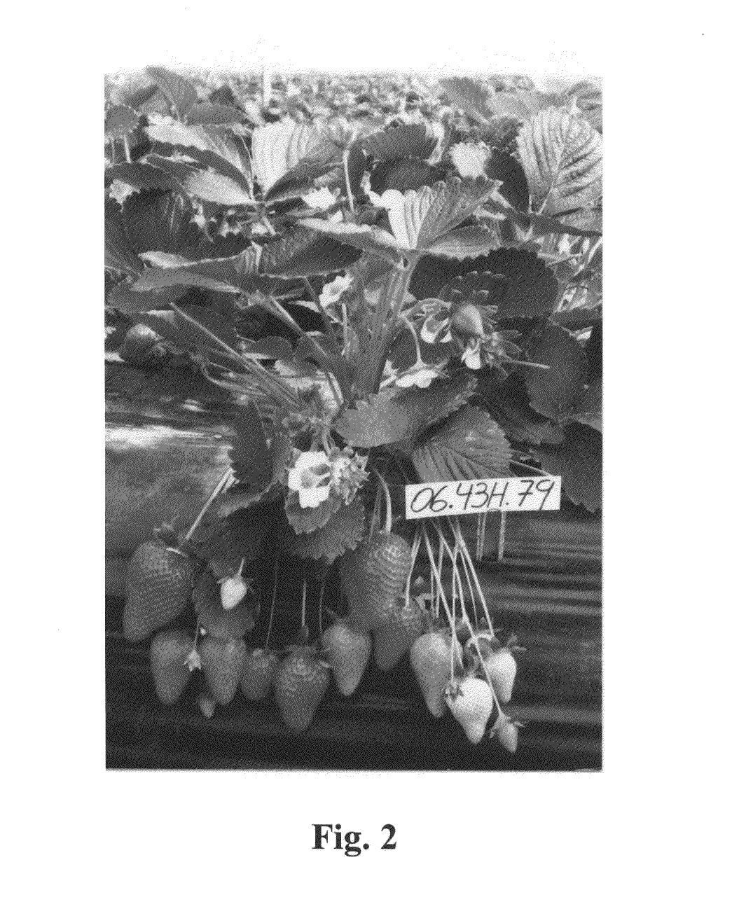

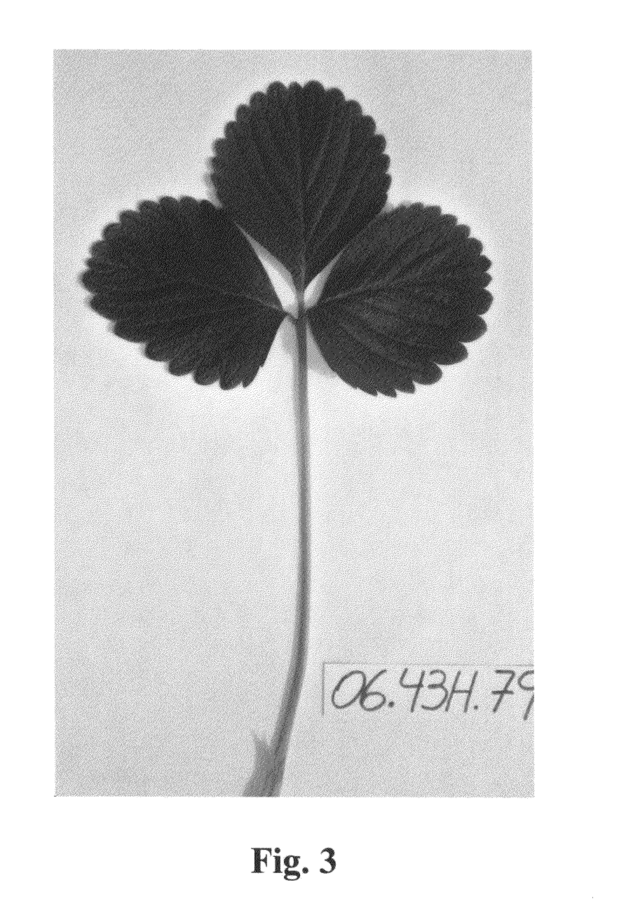

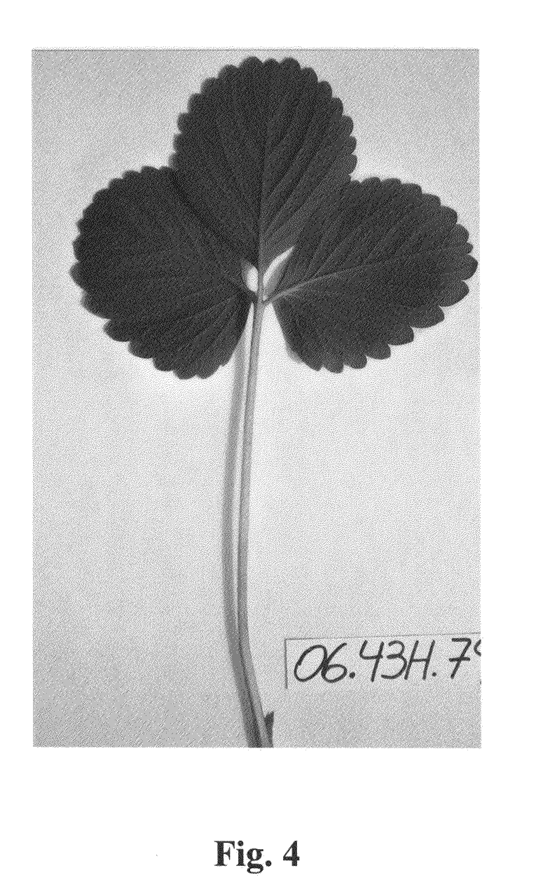

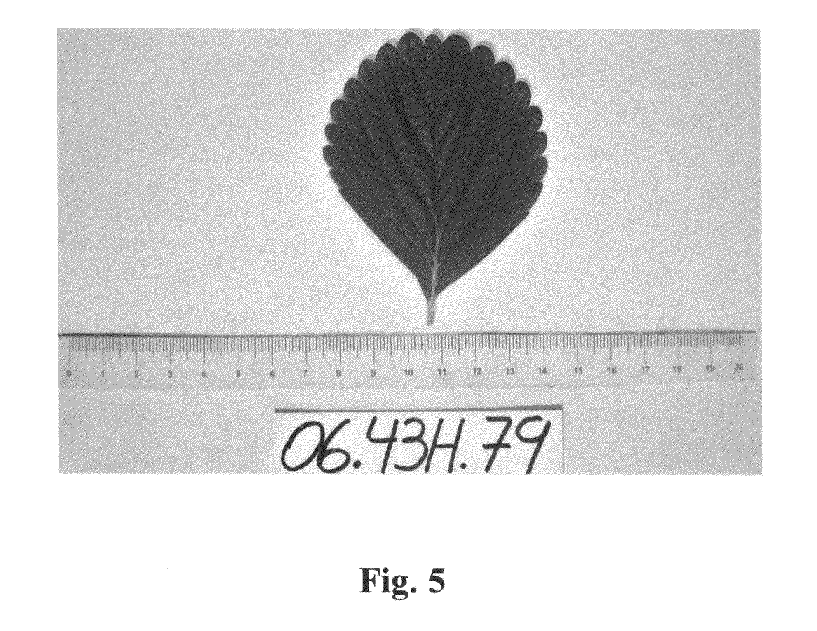

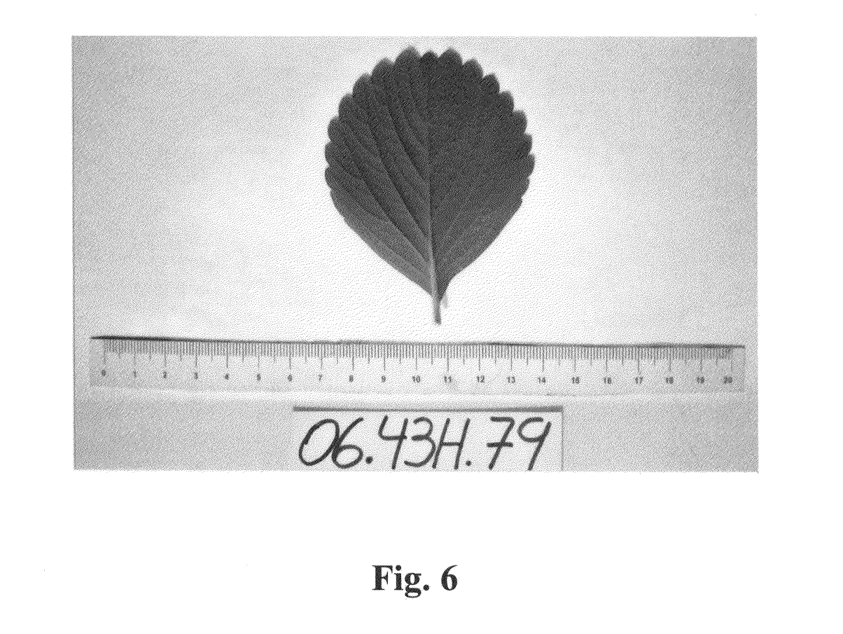

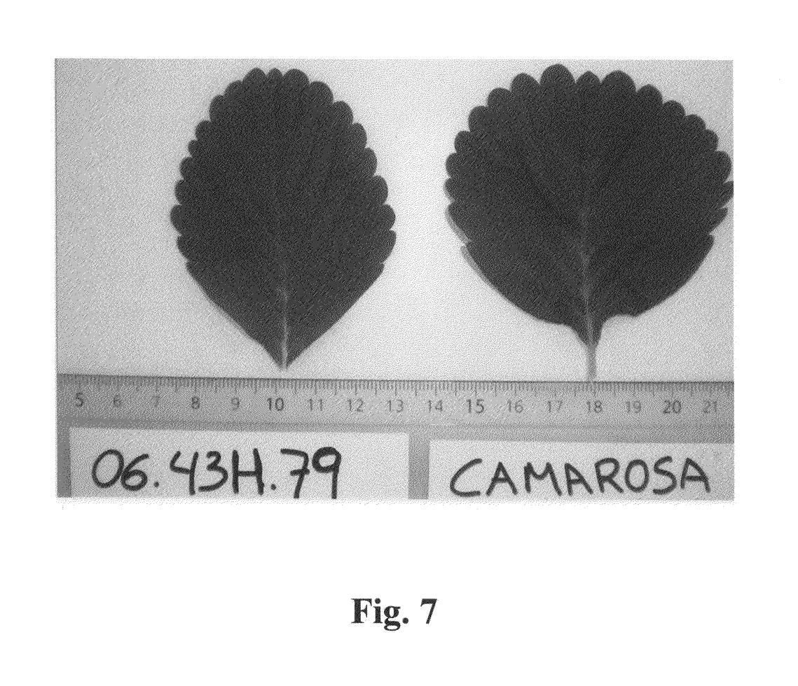

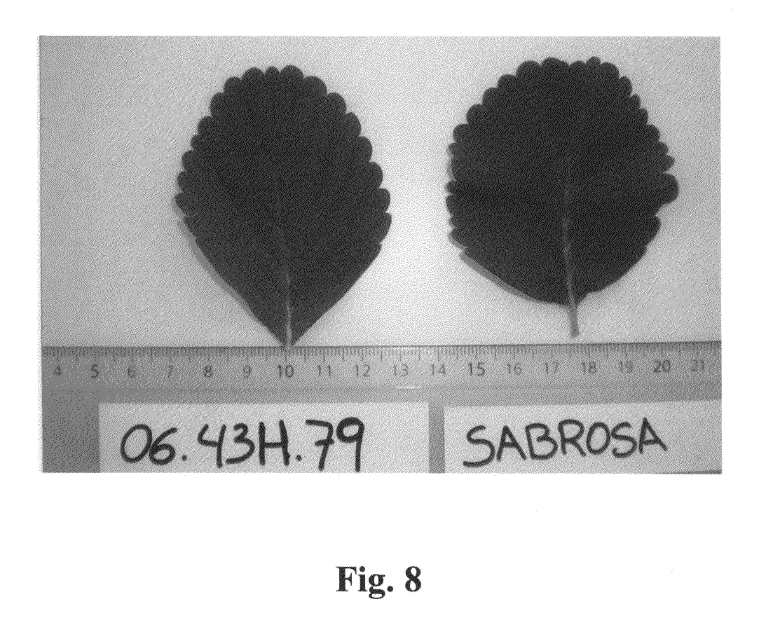

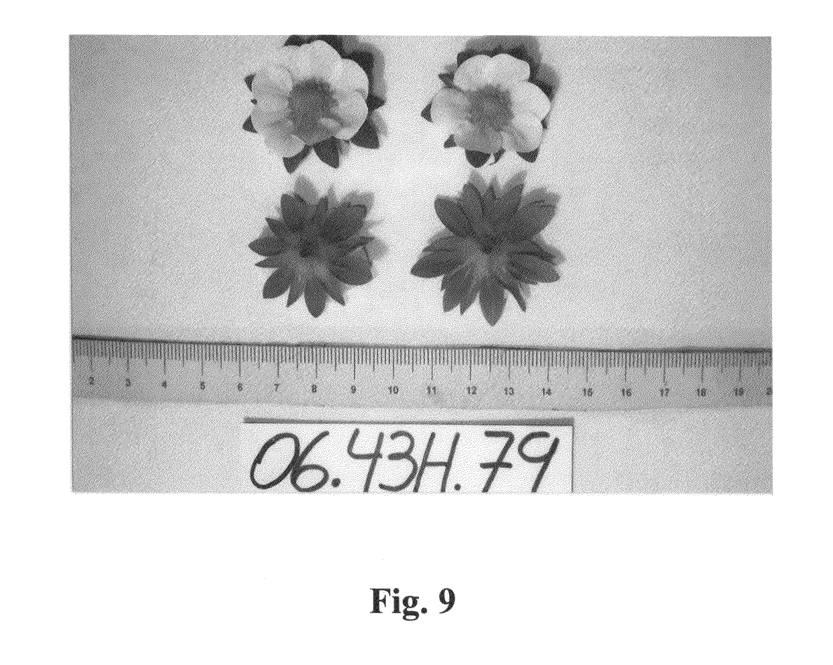

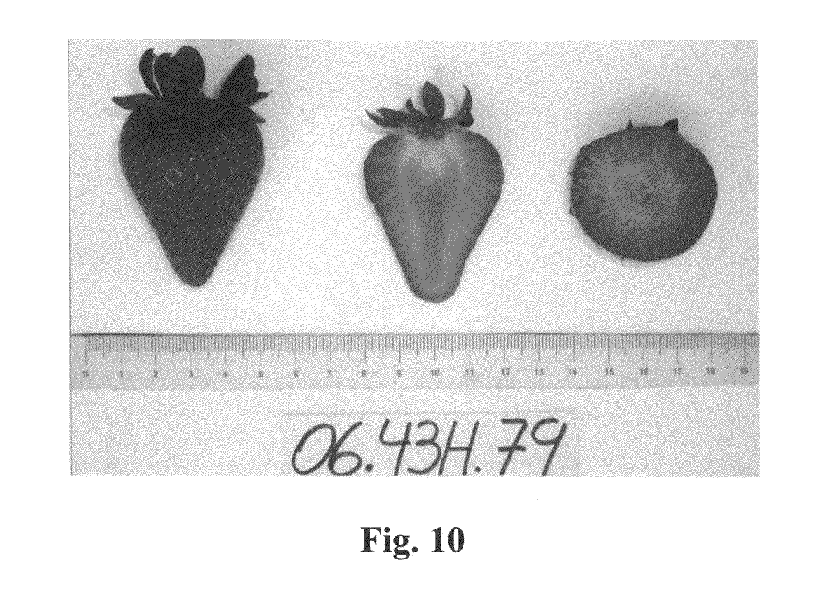

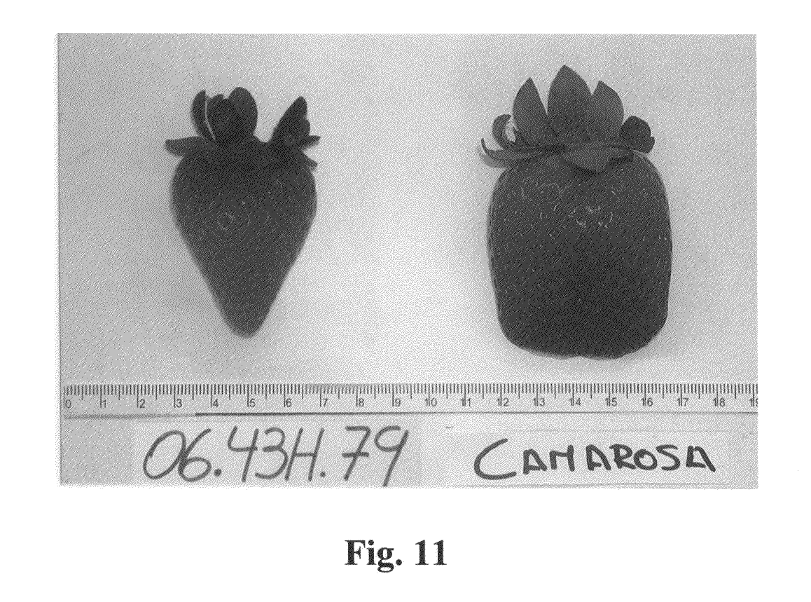

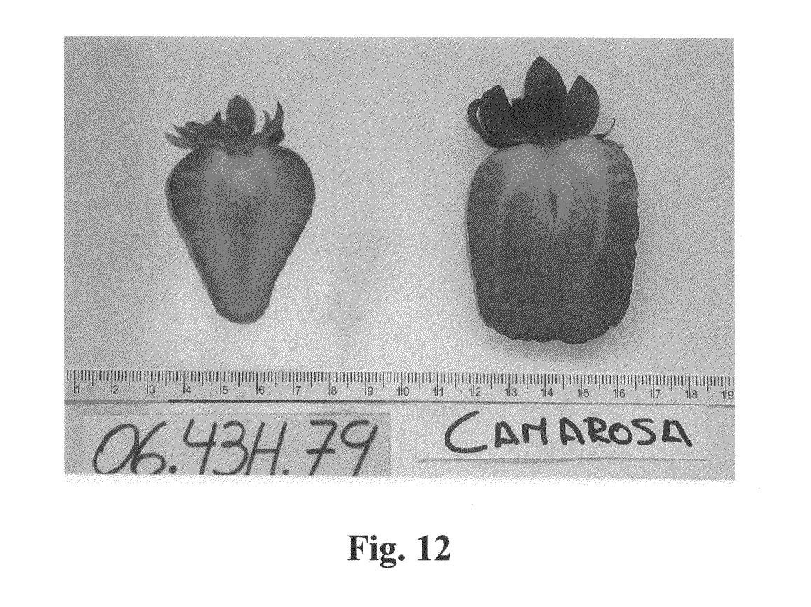

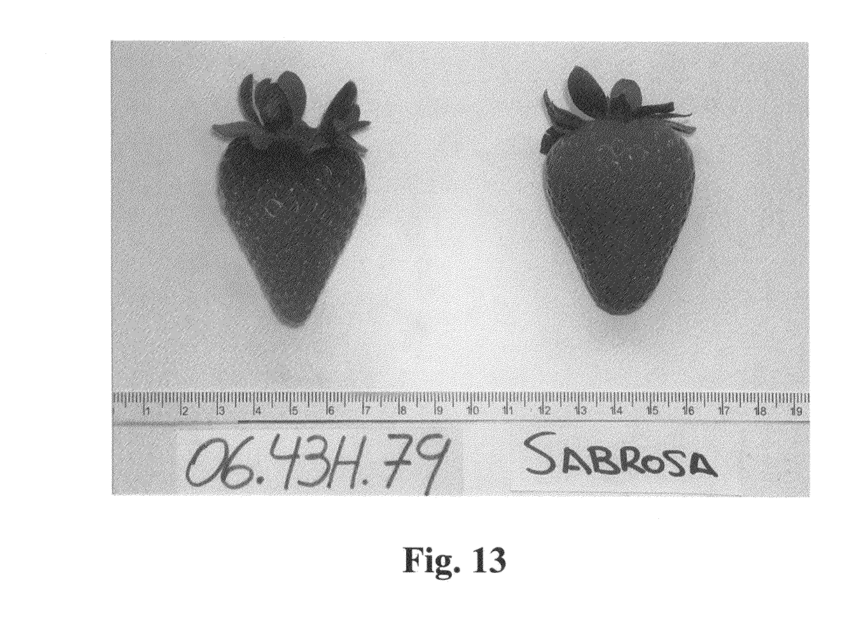

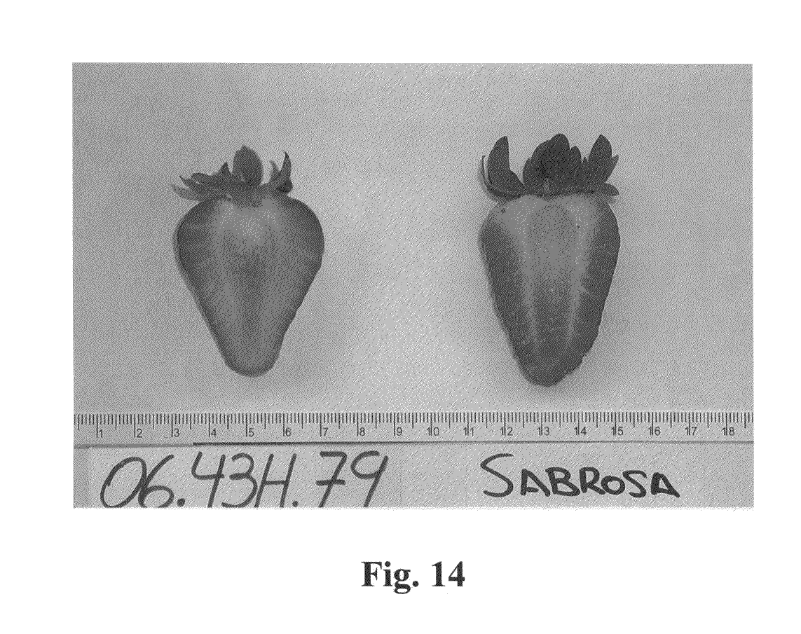

### DESCRIPTION OF THE NEW VARIETY

Throughout this specification, color names beginning with a small letter signify that the name of that color, as used in common speech is aptly descriptive. Color names beginning with a capital letter designate values based upon the R.H.S. Colour Chart published by The Royal Horticultural Society, London, England, 1995. The color descriptions and other phenotypical descriptions may deviate from the stated values and descriptions depending upon variation in environmental, seasonal, climatic and cultural conditions.

The following The following detailed description of the new variety is based upon observations taken of plants and fruits grown in Cartaya (Huelva), Spain, 7° W, 37° N, 45 feet elevation.

The new variety is principally propagated by way of runners. Although propagation by runners is presently preferred, other known methods of propagating strawberry plants may be used. Strawberries root well after transplanting.

The term “blistering” used herein refers to the texture or rugosity or surface ondulation inherent to leaves and is generally a constant characteristic.

Table 1 shows the Weight (g/Fruit) at March 18 of the new variety “Sahara” when compared its closest variety “Sabrosa,” and another variety, “Camarosa.”

TABLE 1

| WEIGHT (g/fruit)ⁱ | March 18 |
|---|---|
| Sahara | 37.9 |
| Sabrosa | 39.3 |
| Camarosa | 24.7 |

ⁱWEIGHT is shown as the average weight per fruit in First Quality Fruits.

Table 2 shows a comparison of the fruit analysis on March 18 between the new variety “Sahara,” its closest variety, “Sabrosa,” and another variety, “Camarosa.”

TABLE 2

| | SAHARA | SABROSA | CAMAROSA |
|---|---|---|---|
| Humidity & Volatile Matter (%) | 91.0 | 91.2 | 90.4 |
| Dry Matter (%) | 9.0 | 8.8 | 9.6 |
| pH (to 20°) | 3.8 | 3.7 | 3.7 |
| Acidity as Anhydride Citric (%) | 0.6 | 0.6 | 0.6 |
| Soluble solids (°Brix) | 7.9 | 7.0 | 7.5 |
| Maturity Index | 13.1 | 11.7 | 12.5 |
| Dominant Tonality (nm) | 490 | 495 | 495 |
| Luminosity: Transmittance | 18.6 | 27.3 | 31.5 |
| to 460 nm | | | |

Dry Matter: It is the weight of the residual left from the trituration of the fruit after the drying process at a temperature of 103° C. + 2° C. until reaching constant weight.

$$\% Dry Matter = \frac{Weight Dry Matter}{Weight Fresh Matter} \times 100$$

Humidity & Volatile Matter: Represents the content in volatile matters and water of the fruits.

### (%) Humidity & Volatile Matter=100−% Dry Matter

Maturity Index: Relation between Soluble solids and Acidity as Anhydride Citric.

$$Maturity Index = \frac{Soluble solids}{Acidity as Anhydride Citric}$$

### DETAILED DESCRIPTION OF THE NEW VARIETY

Plant:
   Growth habit.—Upright.
   Habit.—Globose.
   Density.—Open.
   Vigor.—Medium.
Leaf:
   Size.—Large.
   Upperside.—Medium Green.
   Cross section.—Slightly concave to flat.
   Leaf surface ondulation or blistering.—Weak.
   Glossiness.—Medium.
   Leaf variegation.—Absent.
Terminal leaflet:
   Length/width ratio.—Longer than broad.
   Shape in cross section.—Slightly concave to flat.
   Shape of base.—Acute.
   Margin.—Crenate.
Petiole:
   Attitude of hairs.—Slightly outwards.
   Length.—Long (about 12cm).
Stipule:
   Anthocyanin coloration.—Weak.
   Size.—Medium.
Stolons:
   Number.—Medium.
   Antocyanin coloration.—Weak.
   Pubescence.—Weak.
Inflorescence:
   Position relative to foliage.—Level with.
   Number of flowers.—Medium.
Flower:
   Size.—Medium.
   Size of calyx relative to corolla.—Same size.
   Stamen.—Present.
Primary flower:
   Relative positioning of petals.—Overlapping.
Petal:
   Length/width ratio.—Broader than long.
   Color.—White.
Fruit:
   Ratio of length/width.—Slightly longer than broad.
   Color.—Red.
   Size.—Medium.
   Difference in shapes between primary and secondary fruits.—Moderate.
   Band without achenes.—Absent or very narrow.
   Unevenness of surface.—Weak.
   Evenness of colour.—Even.
   Glosiness.—Medium.
   Insertion of achenes.—Below surface.
   Insertion of calyx.—Level with fruit.
   Attitude of the calyx segments.—Reflexed.
   Size of the calyx in relation to fruit diameter.—Slightly smaller.
   Adherence of calyx.—Strong.
   Firmness.—Firm.
   Colour of flesh.—Orange Red.
   Hollow center.—Weakly expressed.
   Color of core.—Light red.
   Distribution of red colour of flesh.—Only marginal.
   Time of flowering.—Early.
   Tim of ripening.—Early.
   Type of bearing.—Not remontant.
   Shape.—Conical.
Disease resistance: No particular sensitivity to any disease or parasite has been observed for “Sahara”.

What is claimed is:

1. A new and distinct strawberry plant of the variety substantially as shown and described.
